(19) United States
(12) Patent Application Publication
(10) Pub. No.: US 2008/0200753 A1
(43) Pub. Date: Aug. 21, 2008

(54) Electrically operable incontinence treatment apparatus

(75) Inventor: Peter Forsell, Zug (CH)

Correspondence Address: NIXON & VANDERHYE, PC, 901 NORTH GLEBE ROAD, 11TH FLOOR, ARLINGTON, VA, 22203, US

(73) Assignee: Potencia Medical AG, Zugerstrasse (CH)

(21) Appl. No.: 12/081,544

(22) Filed: Apr. 17, 2008

Related U.S. Application Data
(63) Continuation of application No. 10/536,420, filed on Oct. 25, 2005, now abandoned.

(51) Int. Cl. A61F 5/41; A61F 5/00

(52) U.S. Cl. 600/38; 600/30

(57) ABSTRACT

A male sexual impotence treatment apparatus comprises a constriction device (3,6) implanted in a male patient, who suffers from sexual impotence, and engaging the patient's penile tissue or the prolongation thereof, and an electrically powered operation device, which is integrated with the constriction device and adapted to operate the latter to temporarily constrict the penile tissue or the prolongation thereof to restrict the penile exit blood flow to achieve erection. An electric power supply (14) is subcutaneously implanted in the patient remote from the constriction device and an insulated electric wire (18) connects the power supply and the electrically powered operation device. The electric wire is resilient and extends helically between the electric power supply and the electrically powered operation device. As a result, the resilient helically extending electric wire (18) is capable of addressing the movements from the penile tissue or the prolongation thereof, because it may easily be temporarily extended without risking breakage.

The present invention relates to a male sexual impotence treatment apparatus, comprising a constriction device implantable in a male patient, who suffers from sexual impotence, for engaging the patient's penile tissue or the prolongation thereof, and an implantable electrically powered operation device that operates the constriction device to temporarily constrict the penile tissue or the prolongation thereof to restrict the penile exit blood flow to achieve erection. The apparatus further comprises an electric power supply adapted to be implanted in the patient remote from the operation device, and an insulated electric wire connecting the electric power supply and the operation device.

The expression “penile tissue or the prolongation thereof” should be understood to mean the penile tissue extended inside the human body and following the pathway of the blood flow leaving the penis i.e. one or more exit veins from the penis, the corpus cavernosum, crura or the prolongation thereof.

Male sexual impotence is a widespread problem. Many different solutions to this problem have been tried. A main solution currently practised and disclosed in for instance U.S. Pat. Nos. 5,437,605 and 4,841,461 is to implant a hydraulic inflatable silicone prosthesis in the cavities of the corpora cavernosa of the patient's penis. In fluid connection with this prosthesis is a reservoir implanted in the scrotum. By manual pumping action the prosthesis is filled with fluid from the reservoir to effect erect penile condition or is emptied of fluid, which returns to the reservoir, to effect flaccid penile condition. However, there are several more or less severe disadvantages of this main solution. Above all, the penis is more or less damaged by the operation and it is practically impossible to reverse the operation. Another disadvantage is that rather strong forces act against this implanted prosthesis resulting in a significant risk of the prosthesis being broken.

Another solution to achieve erection is to restrict the blood flow leaving the penis. For example, U.S. Pat. No. 4,829,990 discloses two hydraulically operated inflatable cuffs wrapped around the respective crura. A disadvantage of such a solution is that it involves complicated surgery. Another example on this solution is given by U.S. Pat. No. 4,828,544, which discloses an artificial fistula system surgically implanted and providing a primary fistula between the femoral artery and the femoral vein and a secondary fistula for leading blood from the primary fistula to the penis. An inflatable balloon engages the primary fistula between the secondary fistula and the vein. The balloon is in fluid connection with a manually compressible reservoir implanted in the scrotum. Again, implantation of this artificial fistula system requires delicate surgery.

Yet another example on the blood flow restriction solution is given by WO 01/54626, which discloses an elongate hydraulically adjustable constriction member adapted to temporarily constrict the penile tissue or the prolongation thereof of a patient's penile tissue to restrict the blood flow leaving the penis to achieve erection. There are an adjustment device that includes an inflatable cavity in the elongate constriction member and hydraulic means for adding hydraulic fluid to and withdrawing hydraulic fluid from the inflatable cavity.

A mechanical apparatus for constricting the penile tissue or the prolongation thereof may typically involve an electrically powered operation device connected to the constriction device. Therefore, such an operation device would be located at the patient's penile tissue. On the other hand, the electric power supply, for example a battery, necessary for powering the operation device with electric energy should be implanted subcutaneously at a suitable location, which would be remote from the operation device, in order to permit easy access from outside the patient's body for service or replacement of the power supply. In consequence, it would be necessary to implant a fairly long electric wire for connecting the power supply to the operation device. However, considering the fact that the apparatus is to be implanted for many years, possibly for the rest of the patient's life, the use of such an electric wire would make the apparatus unreliable, because the movements of the penile tissue or the prolongation thereof might sooner or later break the electric wire.

The object of the present invention is to provide a new reliable electrically operable apparatus for constricting the penile tissue or the prolongation thereof of an impotent patient.

This object is obtained by an apparatus of the kind presented initially characterised in that the electric wire is resilient and extends helically between the electric power supply and the electrically powered operation device. As a result, the resilient helically extending electric wire is capable of addressing the movements from the penile tissue or the prolongation thereof, because it may easily be temporarily extended without risking breakage.

The constriction device may be implanted in the base of the patient's penis or the prolongation thereof and preferably may engage the corpus cavernosum, crura or the prolongation thereof of the penis. However, there are several alternative positions of the constriction device that give more or less satisfactory restriction of the blood flow leaving the penis. Thus, as a first alternative the constriction device may extend around both corpora cavernosa or crura of the penis as a single unit. As a second alternative the constriction device may comprise two elongated constriction members extending around the respective corpora cavernosa or crura. As a third alternative an elongated constriction member of the constriction device may encircle one or more of the penile exit veins. As a fourth alternative the constriction device may comprise several constriction members extending around the respective penile exit veins.

The electric wire should be designed to conduct a current in the order of milliamperes, which is needed for powering the operation device when operating the constriction device, so that the constriction device exerts a force strong enough to constrict the penile tissue or the prolongation thereof, so that the patient's penile exit blood flow is restricted. Thus, the electric wire should have a low resistance.

Advantageously, the apparatus may include an energy transforming device capable of transforming wireless energy transmitted from outside the patient's body into electric energy, whereby the need for regularly replacing the electric power supply, when it is depleted, is eliminated.

Preferably, the operation device includes an electric motor connected to the electric power supply by means of the helical electrical wire. The constriction device may include a hydraulic constriction device inflatable by hydraulic fluid, and the operation device may include an electrically driven pump (for example driven by the electric motor) hydraulically connected to the hydraulic constriction device.

In accordance with a preferred embodiment of the invention, the constriction device is non-inflatable. In this embodiment, the constriction device comprises an elongate constriction member adapted to extend around the penile tissue or the prolongation thereof. The constriction member includes a main portion and two elongated end portions, and the operation device establishes longitudinal relative displacement between the end portions of the constriction member to constrict or release the penile tissue or the prolongation thereof. The operation device may include a movement transferring means in engagement with at least one of the end portions of the constriction member and operable by the electric motor to displace the one end portion relative to the other end portion of the constriction member. The movement transferring means may include a gear wheel fixed to the other end portion of the constriction member and a gear rack formed on the one end portion of the constriction member, the gear wheel and the gear rack being in mesh with each other. The operation device may further include a worm gear connected between the electric motor and the gear wheel.

Generally, the constriction device includes a housing containing the operation device, and the elongate constriction member and the housing are adapted to form a loop around the patient's penile tissue or the prolongation thereof.

Advantageously, the apparatus includes a control device, suitably in the form of a wireless remote control, for controlling the operation device to operate the constriction device. The control device may include an internal, preferably programmable, control unit implantable in the patient for controlling the operation device. Furthermore, the control device may include an external control unit outside the patient's body, wherein the internal control unit is programmable by the external control unit.

The apparatus may include at least one implantable sensor for sensing at least one physical parameter of the patient. The sensor may be a pressure sensor for directly or indirectly sensing the pressure against the constriction device. The control device may suitably control the operation device in response to signals from the sensor. Where the control device includes an internal control unit, the internal control unit may control the operation device in response to signals from the sensor.

The apparatus may include a switch implantable in the patient for directly or indirectly switching the electric energy supplied by the electric power supply.

In the enclosed drawings:

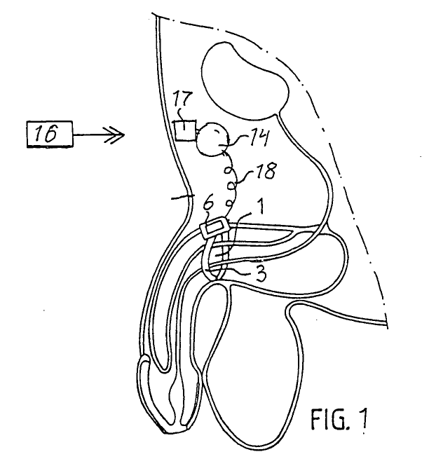

FIG. 1 is a schematic view of an apparatus according to the present invention implanted in a male impotent patient, and

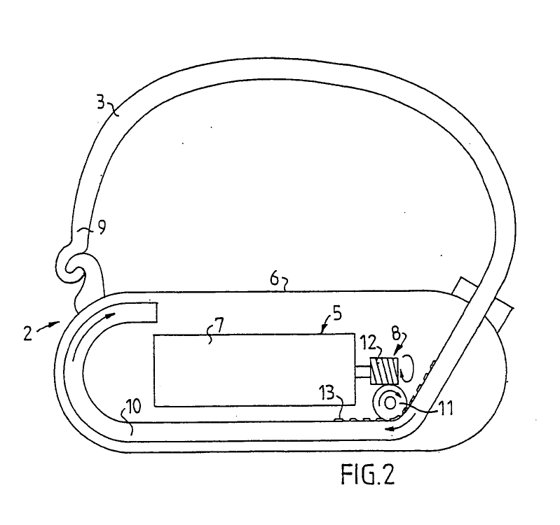

FIG. 2 schematically illustrates an embodiment of a constriction device of the apparatus for use in the embodiment of FIG. 1.

FIG. 1 shows an apparatus of the present invention for treating an impotent patient comprising a constriction device 2 applied on the patient's penile tissue 1. The constriction device 2 includes an elongate operable constriction member 3 extending in a loop around the penile tissue 1. An electrically powered operation device 5, which operates the constriction member 3 to temporarily constrict the penile tissue when erection is desired, is housed in an elongate housing 6, see FIG. 2. The constriction member 3 has a first end portion 9 releasably connected to the housing 6 and a second end portion 10 connected to the operation device 5. The operation device 5 includes an electric motor 7 and a movement transferring means 8 in engagement with the end portion 10. The electric motor 7 operates the movement transferring means 8 to displace the end portion 10 relative to portion 9 in the loop formed by the constriction member 3. The movement transferring means 8 includes a gear wheel 11 fixed to the housing 6, a worm gear 12 connected between the electric motor 7 and the gear wheel 11, and a gear rack 13 formed on the end portion 10, wherein the gear wheel 11 and the gear rack 13 are in mesh with each other.

A rechargeable electric power supply 14 is subcutaneously implanted in the patient. An external remote control 16 controls the operation device 5 and transmits signals that are received by a combined control and energy transforming unit 17 subcutaneously implanted in the patient. The unit 17 is electrically connected to the electric power supply 14 and transforms the energy of the signals into an electric current that is used for charging the electric power supply 14. For example, the signals may include electromagnetic waves and the unit 17 may include an electric p-n junction element that transforms the wireless energy into an electric current.

A resilient insulated electric wire 18 connects the power supply 14 and the electric motor 7. The electric wire 18 extends helically between the power supply 14 and housing 6 that contains the motor 7, in order to permit the electric wire 18 to be temporarily extended when movements of the penile tissue occur, so that the risk of breaking the electric wire 18 is eliminated.

1. A male sexual impotence treatment apparatus, comprising a constriction device implantable in a male patient, who suffers from sexual impotence, for engaging the patient's penile tissue or the prolongation thereof, an implantable electrically powered operation device that operates the constriction device to temporarily constrict the penile tissue or the prolongation thereof to restrict the penile exit blood flow to achieve erection, an electric power supply adapted to be implanted in the patient remote from the operation device, and an insulated electric power wire connecting the electric power supply and the operation device, wherein the electric power wire is resilient and extends helically between the electric power supply and the electrically powered operation devices.

2. The apparatus according to claim 1, further comprising an implantable energy transforming device for transforming wireless energy transmitted from outside the patient's body into electric energy.

3. The apparatus according to claim 1, wherein the operation device comprises an electric motor connected to the electric power supply by means of the helical electrical wire.

4. The apparatus according to claim 3, wherein the constriction device is non-inflatable.

5. The apparatus according to claim 4, wherein the constriction device comprises an elongate constriction member adapted to extend around the penile tissue or the prolongation thereof.

6. The apparatus according to claim 5, wherein the constriction member comprises a main portion and two elongated end portions, and the operation device establishes longitudinal relative displacement between the end portions of the constriction member.

7. The apparatus according to claim 6, wherein the operation device comprises an electric motor and a movement transferring means in engagement with at least one of the end portions of the constriction member and operable by the electric motor to displace the one end portion relative to the other end portion of the constriction member.

8. The apparatus according to claim 7, wherein the movement transferring means comprises a gear wheel fixed relative to the other end portion of the constriction member and a gear rack formed on the one end portion of the constriction member, the gear wheel and the gear rack being in mesh with each other.

9. The apparatus according to claim 8, wherein the operation device comprises a worm gear connected between the electric motor and the gear wheel.

10. The apparatus according to claim 1, wherein the constriction device comprises a hydraulic constriction device and the operation device comprises an electrically driven pump hydraulically connected to the hydraulic constriction device.

11. The apparatus according to claim 5, wherein the constriction device comprises a housing containing the operation device, and the elongate constriction member and the housing are adapted to form a loop around the patient's penile tissue or the prolongation thereof.

12. The apparatus according to claim 1, further comprising a wireless remote control for controlling the operation device to operate the constriction device.

13. The apparatus according to claim 1 further comprising at least one implantable sensor for sensing at least one physical parameter of the patient.

14. The apparatus according to claim 13, wherein the sensor comprises a pressure sensor for directly or indirectly sensing the pressure against the constriction device.

15. The apparatus according to claim 14, further comprising a control device for controlling the operation device in response to signals from the sensor.

16. The apparatus according to claim 15, wherein the control device comprises an internal control unit implantable in the patient for controlling the operation device in response to signals from the sensor.

17. The apparatus according to claim 1, further comprising a control device for controlling the operation device

18. The apparatus according to claim 17, wherein the control device comprises an internal control unit implantable in the patient for controlling the operation device.

19. The apparatus according to claim 18, wherein the internal control unit is programmable.

20. The apparatus according to claim 19, wherein the control device comprises an external control unit outside the patient's body, the internal control unit being programmable by the external control unit.

21. The apparatus according to claim 1, further comprising a switch implantable in the patient for directly or indirectly switching the electric energy supplied by the electric power supply.
